(19) United States
(12) Patent Application Publication
(10) Pub. No.: US 2019/0380762 A1
(43) Pub. Date: Dec. 19, 2019

(54) Blood Vessel Isolation Ablation Device

(71) Applicant: St. Jude Medical, Cardiology Division, Inc., St. Paul, MN (US)

(72) Inventors: Travis Dahlen, Forest Lake, MN (US); Troy Tegg, Elk River, MN (US)

(21) Appl. No.: 16/485,022

(22) PCT Filed: Feb. 21, 2018
(86) PCT No.: PCT/US2018/018953

§ 371 Date: Aug. 9, 2019

Related U.S. Application Data
(60) Provisional application No. 62/461,390, filed on Feb. 21, 2017.

(51) Int. Cl. A61B 18/02; A61B 90/00

(52) U.S. Cl. CPC A61B 18/02; A61B 2018/0022; A61B 90/06

(57) ABSTRACT

A kit for use in cryoablation procedures includes a cryoablation catheter having a cryoballoon positioned about its distal region. A pressure sensor is positioned distally of the cryoballoon. The catheter can be introduced into a patient's left atrium and the cryoballoon inflated. The cryoballoon can then be advanced into contact with a pulmonary vein wall in a manner that occludes the pulmonary vein. Occlusion can be verified by measuring a pressure within the pulmonary vein. Once occlusion is verified, the cryoballoon can be cooled to form a circumferential lesion about the pulmonary vein.

### CROSS-REFERENCE TO RELATED APPLICATIONS

This application claims the benefit of United States provisional application no. 62/461,390, filed 21 Feb. 2017, which is hereby incorporated by reference as though fully set forth herein.

### BACKGROUND

The instant disclosure relates generally to tissue ablation. In particular, the instant disclosure relates to a medical device for use in creating a circumferential lesion about a blood vessel, such as in pulmonary vein (PV) isolation procedures.

In the normal heart, contraction and relaxation of the heart muscle (myocardium) takes place in an organized fashion as electro-chemical signals pass sequentially through the myocardium from the sinoatrial (SA) node, which comprises a bundle of unique cells disposed in the wall of the right atrium, to the atrioventricular (AV) node and then a long a well-defined route, which includes the His-Purkinje system, into the left and right ventricles. Sometimes, however, abnormal rhythms occur in the heart, which are referred to generally as arrhythmias.

Certain arrhythmias, referred to as atrial arrhythmias, occur in the atria. Three of the most common atrial arrhythmias are ectopic atrial tachycardia, atrial fibrillation (AF), and atrial flutter. AF can result in significant patient discomfort and even death because of a number of associated problems, including: irregular heart rate, which causes patient discomfort and anxiety; loss of synchronous atrioventricular contractions, which compromises cardiac hemodynamics, resulting in varying levels of congestive heart failure; and stasis of blood flow, which increases the likelihood of thromboembolism, a leading cause of stroke.

One common medical procedure for the treatment of certain types of cardiac arrhythmia, specifically including AF, is catheter ablation. In many ablation procedures, energy, such as radiofrequency (RF) or high intensity focused ultrasound (HIFU) energy, is delivered to cardiac tissue in order to heat the tissue and create a permanent scar or lesion that is electrically inactive.

It is known that, in some instances, stray electrical signals find a pathway down the pulmonary veins and into the left atrium of the heart. In these instances, it may be advantageous to produce a circumferential lesion at or near the ostium of one or more of the pulmonary veins. Desirably, such a circumferential lesion would electrically isolate the pulmonary vein from the left atrium, completely blocking stray signals from traveling down the pulmonary vein and into the left atrium. Thus, such procedures are known as PV isolation procedures.

PV isolation procedures are often carried out cryogenically. More particularly, PV isolation procedures are often carried out using a cryoballoon, such as found on the Arctic Front Advance™ Cardiac CryoAblation Catheter from Medtronic.

In a cryoballoon procedure, the practitioner should ensure that the cryoballoon is securely in contact with the PV wall, as gaps between the balloon and the vessel wall can complicate lesion creation. It is known to verify such contact by injecting a contrast medium which can be detected via fluoroscopy. Yet, the increased fluoroscopy dosage is not as desirable.

### BRIEF SUMMARY

Disclosed herein is an apparatus for use in ablation procedures, such as cryoablation procedures. The apparatus includes: a ablation catheter (e.g., a cryoablation catheter) including: an elongate body having a lumen, the elongate body including a distal region; and a balloon positioned about the distal region and defining an interior in communication with the lumen, wherein the balloon is expandable outwardly from an outer surface of the elongate body; and a pressure sensor (e.g., an optical pressure sensor) positioned distally of the balloon. According to aspects of the disclosure, the apparatus also includes an electrophysiology catheter (e.g., a circular mapping catheter) dimensioned for insertion through the lumen, with the pressure sensor including at least one pressure sensor positioned on at least one of a distal region of the electrophysiology catheter and the distal region of the ablation catheter. For example, a first pressure sensor can be positioned on the distal region of the electrophysiology catheter and a second pressure sensor can be positioned on the distal region of the ablation catheter.

The apparatus can also include a guidewire over which the ablation catheter can be advanced, and the pressure sensor can include at least one pressure sensor positioned on at least one of a distal region of the guidewire and the distal region of the ablation catheter. For example, a first pressure sensor can be positioned on the distal region of the guidewire and a second pressure sensor can be positioned on the distal region of the ablation catheter.

In embodiments disclosed herein, a pressure sensor can be positioned on the elongate body proximally of the balloon.

Also disclosed herein is a system for use in cryoablation procedures, including: a first device; a second device including: an elongate body having a lumen dimensioned to receive the first device therethrough, the elongate body including a distal region; and a balloon positioned about the distal region and defining an interior in communication with the lumen, wherein the balloon is expandable outwardly from an outer surface of the elongate body; and at least one pressure sensor (e.g., an optical pressure sensor) positioned on at least one of the distal region of the second device and a distal region of the first device. The first device can be a guidewire, an electrophysiology catheter, or the like.

The at least one pressure sensor can include a first pressure sensor positioned on the distal region of the first device and a second pressure sensor positioned on the distal region of the second device. In some embodiments of the disclosure, the at least one pressure sensor is positioned on the distal region of the second device distally of the balloon.

It is also contemplated that the system can include at least one pressure sensor positioned on the second device proximally of the balloon.

The instant disclosure also provides a method of performing a pulmonary vein isolation. The method includes: introducing an ablation catheter (e.g., a cryoablation catheter) into a left atrium of a heart, the ablation catheter including a balloon; inflating the balloon; advancing the balloon into contact with a wall of a pulmonary vein; verifying that the balloon is occluding the pulmonary vein by measuring a pressure within the pulmonary vein; and delivering an ablation fluid to the balloon to form a circumferential lesion about the pulmonary vein, such as by cooling or heating the cryoballoon. The pressure within the pulmonary vein can be measured by using a pressure sensor mounted to the ablation catheter distal of the balloon.

According to aspects of the disclosure, the step of introducing an ablation catheter into a left atrium of a heart includes introducing the ablation catheter into the left atrium of the heart over a guidewire; and the step of measuring the pressure within the pulmonary vein includes measuring the pressure within the pulmonary vein using a pressure sensor mounted to a region of the guidewire that remains distal of the balloon after introducing the ablation catheter into the left atrium of the heart over the guidewire.

In other aspects of the disclosure, the method can also include introducing an electrophysiology catheter into the pulmonary vein through a lumen in the ablation catheter, and measuring the pressure within the pulmonary vein can include measuring the pressure within the pulmonary vein using a pressure sensor mounted to a region of the electrophysiology catheter that extends distally of the ablation catheter.

The step of verifying that the balloon is occluding the pulmonary vein can include: measuring a pressure within the pulmonary vein using a first pressure sensor positioned distal of the balloon; measuring a pressure within the left atrium using a second pressure sensor positioned proximal of the balloon; and comparing the pressure within the pulmonary vein to the pressure within the left atrium.

In yet another embodiment, the instant disclosure relates to a method of performing a pulmonary vein isolation that includes: verifying occlusion of a pulmonary vein by a balloon utilizing a pressure within the pulmonary vein; and delivering ablation therapy to the pulmonary vein after verifying occlusion of the pulmonary vein. For example, cryoablation therapy can be delivered to the pulmonary vein after verifying occlusion thereof.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a kit for use in a cryoablation procedure.

FIG. 2 depicts details of a cryoablation catheter according to aspects of the instant disclosure.

FIG. 3 depicts details of an electrophysiology catheter according to aspects of the instant disclosure.

FIGS. 4A and 4B depict the conduct of a pulmonary vein isolation procedure.

FIGS. 5A and 5B depict pressure waveforms within the pulmonary vein during a pulmonary vein isolation procedure.

### DETAILED DESCRIPTION

The present disclosure provides medical devices, such as catheters, suitable for use in creating an ablation lesion about the circumference of a blood vessel. Such a lesion is referred to herein as a “circumferential lesion,” and includes both closed-ended lesions (e.g., a circular lesion that loops around the vessel wall a single time) and open-ended lesions (e.g., a helical lesion that loops around the vessel wall multiple times). In particular, the devices disclosed herein allow a practitioner to verify occlusion of the blood vessel, and therefore good contact between the device and the vessel, prior to cryoablation without requiring the introduction of contrast medium.

For purposes of illustration, certain embodiments will be described in the context of a pulmonary vein isolation (“PVI”) cryoablation procedure. It should be understood, however, that the teachings herein can be applied to good advantage in other contexts. For example, the teachings herein are applicable to various balloon-based PVI procedures where direct contact with the vessel wall is required, such as procedures that utilize radiofrequency (“RF”) energy to create ablation lesions.

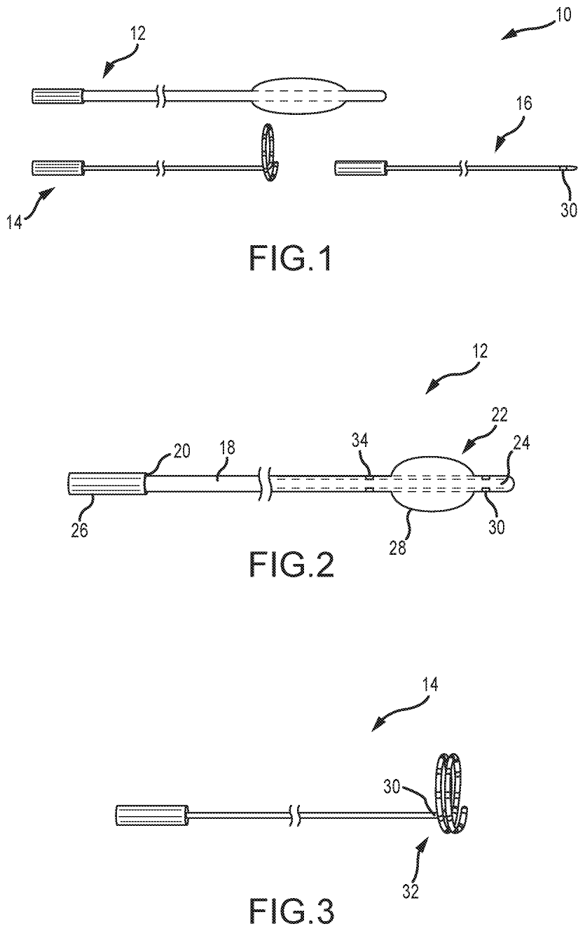

FIG. 1 depicts apparatus 10 for use in cryoablation procedures. As shown in FIG. 1, apparatus 10 generally includes a cryoablation catheter 12, an electrophysiology catheter 14, and a guidewire 16 over which cryoablation catheter 12 can be advanced through a patient's vasculature. Apparatus 10 can also include devices to facilitate a transseptal approach to the left atrium, such as an introducer and a transseptal puncture needle. Those of ordinary skill in the art will have a basic familiarity with the foregoing components of apparatus 10, such that they will be described herein only to the extent necessary to understand the instant disclosure.

As shown in FIG. 2, cryoablation catheter 12 generally includes an elongate body 18 having a proximal end 20 and a distal region 22. Body 18 includes one or more lumens 24 (shown in phantom in FIG. 2). Although only a single lumen 24 is depicted in FIG. 2 for purposes of illustration, it should be understood that any number and configuration of lumens 24 can be used without departing from the scope of the instant teachings. As the person of ordinary skill in the art will appreciate, body 18 can also contain electrical interconnect wires, pull-wires, and the like.

Proximal end 20 of body 18 is attached to a catheter control handle 26. Catheter control handle 26 can include, for example, an actuator coupled to suitable structure (e.g., pull wires and/or pull rings) within body 18 in order to effect the deflection of distal region 22 in one or more bending planes. It can also include electrical power, electrical signal, and/or fluidic connections (e.g., connection to a source of cryogenic fluid).

A balloon 28 is positioned about distal region 22. The interior of balloon 28 is in communication with lumen 24, for example through one or more vias. Thus, a cryogenic fluid can be provided from a fluid source, through lumen 24, in order to expand balloon 28 outwardly from an outer surface of body 18 as well as to cryogenically ablate adjacent tissue.

FIG. 3 depicts electrophysiology catheter 14 in further detail. As shown in FIG. 3, electrophysiology catheter 14 can be a multi-electrode circular mapping catheter similar to the Inquiry™ Optima™ Diagnostic Catheter of St. Jude Medical, Inc. Electrophysiology catheter 14 is dimensioned to fit through lumen 24 of cryoablation catheter 12 as discussed below.

As shown in FIGS. 2 and 3, one or more pressure sensors 30 are mounted on cryoablation catheter 12 and/or electrophysiology catheter 14. In some embodiments, pressure sensor 30 is mounted on cryoablation catheter 12 distally of balloon 28. Thus, when cryoablation catheter 12 is placed for a PVI procedure, pressure sensor 30 can measure the pressure within the pulmonary vein.

Alternatively or additionally, pressure sensor 30 can be mounted on the distal region 32 of electrophysiology catheter 14. Although this pressure sensor 30 is shown as being mounted to the relatively straight portion of the distal region 32 of electrophysiology catheter 14, it could also be mounted within the looped portion of the distal region 32 of electrophysiology catheter 14 without departing from the scope of the instant teachings. In general, however, positioning pressure sensor 30 on the distal region 32 of electrophysiology catheter 14 will allow it to reside within the pulmonary vein when electrophysiology catheter 14 is advanced through lumen 24 of cryoablation catheter 12 as described below.

It is also contemplated to include one or more pressure sensors 34 on cryoablation catheter 12 positioned proximally of balloon 28. This allows for differential pressure readings both inside and outside the pulmonary vein.

Various types of sensors can be used as pressure sensor(s) 30 and 32. In some embodiments, for example, pressure sensor(s) 30 and/or 32 are optical pressure sensors, such as offered by the Technobis group. In other embodiments, pressure sensor(s) 30 and/or 32 can be printed pressure sensors, pressure-sensitive conductive composite (“PSCC”) sensors, strain gauges, pressure sensitive capacitive elements residing within resonant LC circuits, and the like.

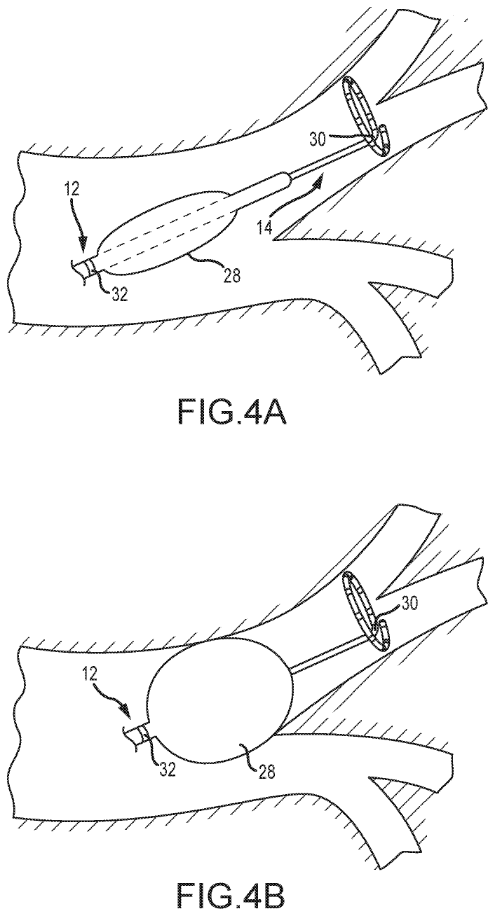

In use, cryoablation catheter 12 is introduced into the left atrium of a patient's heart, for example using a transseptal approach that will be familiar to those of ordinary skill in the art. In the embodiment shown in FIG. 4A, cryoablation catheter 12 is introduced over electrophysiology catheter 14. It should be understood, however, that it could also be introduced over guidewire 16 (for example, where the practitioner does not wish to map the pulmonary vein during the procedure).

Balloon 28 can then be inflated and advanced into contact with the wall of a pulmonary vein as shown in FIG. 4B. As discussed above, it is desirable to ensure that the pulmonary vein is completely occluded by balloon 28 in order to facilitate the creation of a circumferential lesion about the pulmonary vein. Thus, the pressure within the pulmonary vein can be measured using pressure sensor 30 (shown on electrophysiology catheter 14 in FIGS. 4A and 4B; as described above, however, it could also be carried by cryoablation catheter 12).

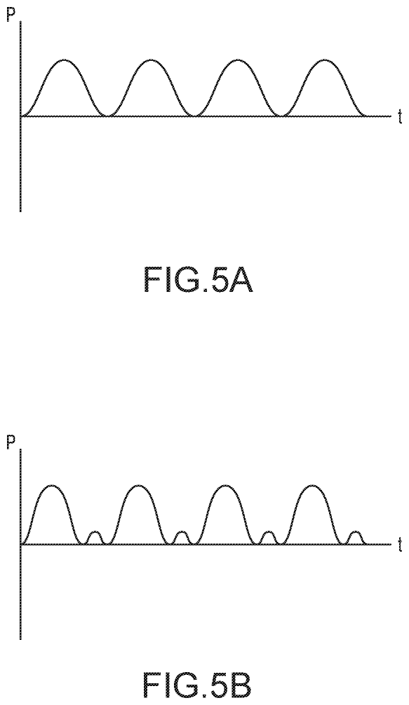

If balloon 28 is completely occluding the pulmonary vein, then the pressure signal from pressure sensor 30 will resemble the trace shown in FIG. 5A. The practitioner can then initiate cooling of the cryoballoon to create the circumferential lesion about the pulmonary vein.

If, on the other hand, there is a leak between balloon 28 and the pulmonary vein, then the pressure signal from pressure sensor 30 will resemble the trace shown in FIG. 5B. If the practitioner observes a signal resembling FIG. 5B, therefore, the practitioner should reposition balloon 28 before cooling the cryoballoon in order to create the circumferential lesion about the pulmonary vein.

In still other embodiments, occlusion van be verified by comparing the pressure measured within the pulmonary vein using pressure sensor 30 to the pressure measured outside the pulmonary vein/in the atrium using pressure sensor 32.

Although several embodiments of this invention have been described above with a certain degree of particularity, those skilled in the art could make numerous alterations to the disclosed embodiments without departing from the spirit or scope of this invention.

For example, a pressure sensor 30 can also be included within the distal region of guidewire 16 (see FIG. 1).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An apparatus for use in ablation procedures, comprising:
an ablation catheter comprising:
an elongate body having a lumen, the elongate body including a distal region; and
a balloon positioned about the distal region and defining an interior in communication with the lumen, wherein the balloon is expandable outwardly from an outer surface of the elongate body; and
a pressure sensor positioned distally of the balloon.

2. The apparatus according to claim 1, further comprising an electrophysiology catheter dimensioned for insertion through the lumen, and wherein the pressure sensor comprises at least one pressure sensor positioned on at least one of a distal region of the electrophysiology catheter and the distal region of the ablation catheter.

3. The apparatus according to claim 2, wherein a first pressure sensor is positioned on the distal region of the electrophysiology catheter and a second pressure sensor is positioned on the distal region of the ablation catheter.

4. The apparatus according to claim 2, wherein the electrophysiology catheter comprises a circular mapping catheter.

5. The apparatus according to claim 1, further comprising a guidewire over which the ablation catheter can be advanced, and wherein the pressure sensor comprises at least one pressure sensor positioned on at least one of a distal region of the guidewire and the distal region of the ablation catheter.

6. The apparatus according to claim 5, wherein a first pressure sensor is positioned on the distal region of the guidewire and a second pressure sensor is positioned on the distal region of the ablation catheter.

7. The apparatus according to claim 1, further comprising a pressure sensor positioned on the elongate body proximally of the balloon.

8. The apparatus according to claim 1, wherein the pressure sensor comprises an optical pressure sensor.

9. A system for use in cryoablation procedures, comprising:
a first device;
a second device comprising:
an elongate body having a lumen dimensioned to receive the first device therethrough, the elongate body including a distal region; and
a balloon positioned about the distal region and defining an interior in communication with the lumen, wherein the balloon is expandable outwardly from an outer surface of the elongate body; and
at least one pressure sensor positioned on at least one of the distal region of the second device and a distal region of the first device.

10. The system according to claim 9, wherein the first device comprises a guidewire.

11. The system according to claim 9, wherein the first device comprises an electrophysiology catheter.

12. The system according to claim 9, wherein the at least one pressure sensor comprises a first pressure sensor positioned on the distal region of the first device and a second pressure sensor positioned on the distal region of the second device.

13. The system according to claim 9, wherein the at least one pressure sensor is positioned on the distal region of the second device distally of the balloon.

14. The system according to claim 9, further comprising at least one pressure sensor positioned on the second device proximally of the balloon.

15. The system according to claim 9, wherein the at least one pressure sensor comprises an optical pressure sensor.

16. A method of performing a pulmonary vein isolation, comprising:
introducing an ablation catheter into a left atrium of a heart, the ablation catheter comprising a balloon;
inflating the balloon;
advancing the balloon into contact with a wall of a pulmonary vein;
verifying that the balloon is occluding the pulmonary vein by measuring a pressure within the pulmonary vein; and
delivering an ablation fluid into the balloon to form a circumferential lesion about the pulmonary vein.

17. The method according to claim 16, wherein measuring the pressure within the pulmonary vein comprises measuring the pressure within the pulmonary vein using a pressure sensor mounted to the ablation catheter distal of the balloon.

18. The method according to claim 16, wherein:
introducing an ablation catheter into a left atrium of a heart comprises introducing the ablation catheter into the left atrium of the heart over a guidewire; and
measuring the pressure within the pulmonary vein comprises measuring the pressure within the pulmonary vein using a pressure sensor mounted to a region of the guidewire that remains distal of the balloon after introducing the ablation catheter into the left atrium of the heart over the guidewire.

19. The method according to claim 16, further comprising introducing an electrophysiology catheter into the pulmonary vein through a lumen in the ablation catheter, and wherein measuring the pressure within the pulmonary vein comprises measuring the pressure within the pulmonary vein using a pressure sensor mounted to a region of the electrophysiology catheter that extends distally of the ablation catheter.

20. The method according to claim 16, wherein verifying that the balloon is occluding the pulmonary vein comprises:
measuring a pressure within the pulmonary vein using a first pressure sensor positioned distal of the balloon;
measuring a pressure within the left atrium using a second pressure sensor positioned proximal of the balloon; and
comparing the pressure within the pulmonary vein to the pressure within the left atrium.

21. The method according to claim 16, wherein delivering an ablation fluid into the balloon to form a circumferential lesion about the pulmonary vein comprises delivering a cryogenic fluid into the balloon, thereby cooling the balloon to form the circumferential lesion via cryoablation.

22. A method of performing a pulmonary vein isolation, comprising:
verifying occlusion of a pulmonary vein by a balloon utilizing a pressure within the pulmonary vein; and
delivering ablation therapy to the pulmonary vein after verifying occlusion of the pulmonary vein.

23. The method according to claim 22, wherein delivering ablation therapy to the pulmonary vein comprises delivering cryoablation therapy to the pulmonary vein.
